(19) United States
(12) Patent Application Publication
(10) Pub. No.: US 2016/0341667 A1
(43) Pub. Date: Nov. 24, 2016

(54) RAMAN SPECTROSCOPY FOR BIOPROCESS OPERATIONS

(71) Applicant: AbbVie, Inc., Chicago, IL (US)

(72) Inventors: Natarajan Ramasubramanyan, Chicago, IL (US); Li-Hong Malmberg, Chicago, IL (US); Martin Sternman, Chicago, IL (US)

(21) Appl. No.: 14/989,491

(22) Filed: Jan. 6, 2016

Related U.S. Application Data
(63) Continuation of application No. 13/234,947, filed on Sep. 16, 2011, now abandoned.
(60) Provisional application No. 61/452,978, filed on Mar. 15, 2011.
(60) Provisional application No. 61/384,131, filed on Sep. 17, 2010.

(51) Int. Cl. G01N 21/65; G01N 33/68

(52) U.S. Cl. CPC G01N 21/65; G01N 33/6854; G01N 2201/13

(57) ABSTRACT

A method of characterizing a multi-component mixture for use in a bioprocess operation that includes providing a multi-component mixture standard with pre-determined amounts of known components; performing a Raman Spectroscopy analysis on the multi-component mixture standard; providing a multi-component test mixture from the bioprocess operation; performing a Raman Spectroscopy analysis on the multi-component test mixture; and comparing the analysis of the multi-component mixture standard and the multi-component test mixture to characterize the multi-component test mixture. In one embodiment, the multi-component mixture standard and the multi-component test mixture both comprise one or more of, at least two, at least three of, or each of, a polysaccharide (e.g. sucrose or mannitol), an amino acid (e.g., L-arginine, L-histidine or L-ornithine), a surfactant (e.g. polysorbate 80) and a pH buffer (e.g., a citrate formulation).

This application is claims the benefit of the priority date of U.S. Ser. No. 61/384,131, filed Sep. 17, 2010, and U.S. Ser. No. 61/452,978, filed Mar. 15, 2011, both of which are hereby incorporated by reference in their entirety.

### 1. INTRODUCTION

The present invention relates to methods for employing Raman Spectroscopy for process monitoring and control of bioprocess operations.

### 2. BACKGROUND

Typical monitoring and control for bioprocess operations include in-process tests like pH, conductivity, protein concentration, and osmolality or analytical techniques such as ELISA or HPLC based methods. These methods tend to be either too generic or too cumbersome and time-consuming. Chemical composition of biologics process intermediates is often essential to control and/or to improve consistency or quality of bioprocess operations. There remains a need for methods to test such multi-component mixtures of biologic process intermediates quickly and accurately to provide real-time or near real time assurance of quality and composition.

### 3. SUMMARY

In certain embodiments, the presently disclosed subject matter provides methods of characterizing multi-component mixtures for use in a bioprocess operation that include: providing a multi-component mixture standard with pre-determined amounts of known components; performing Raman Spectroscopy analysis on the multi-component mixture standard; providing one or more multi-component test mixtures from the bioprocess operation; performing a Raman Spectroscopy analysis on the multi-component test mixtures; and comparing the analysis of the multi-component mixture standard and the multi-component test mixtures to characterize the multi-component test mixtures. For example, comparing the analysis of the multi-component mixture standard and the multi-component test mixtures to characterize the multi-component test mixtures can include fitting data obtained from the multi-component mixture standard through statistical methods to obtain a calibration model and subsequently using it to determine concentrations in the multi-component test mixtures.

In certain embodiments, the multi-component mixture standard and the multi-component test mixture both comprise one or more of, at least two, at least three of, or each of a saccharide (e.g., mannitol), an amino acid (e.g., L-arginine, methionine, L-histidine, L-ornithine proline, alanine, 1-arginine, asparagines, aspartic acid, glycine, serine, lysine, histidine, and glutamic acid), a surfactant (e.g. polysorbate 80), Tween™ and a pH buffer (e.g., a citrate formulation, a Tris buffer, or an acetate buffer). These formulation mixtures can contain other components such as antimicrobial agents (e.g., benzyl alcohol, chlorobutanol, methyl paraben, propyl paraben, phenol, m-cresol) or chelating agents such as EDTA or other components such as polyols, PEG, etc., or proteins such as BSA, etc., or salts such as sodium chloride, sodium succinate, sodium sulfate, potassium chloride, magnesium chloride, magnesium sulfate, and calcium chloride, or alcohols such as ethanol.

In certain embodiments, a series of multi-component mixture standards with pre-determined amounts of known components can be randomly selected, and a Raman Spectroscopy analysis on the series of multi-component mixture standards is performed. Data processing and principal component methods can ensure reliable predictability. For example, a Partial Least Squares Regression Analysis of the Raman Spectroscopy analysis can be performed on the series of multi-component mixture standards to develop a model (e.g., a calibration curve).

In certain embodiments, the multi-component mixture is a formulation suitable for administration to an animal subject (e.g., a human subject). For example, the multi-component mixture can be a formulation buffer intended to be combined with a biologically active agent (e.g., a monoclonal antibody). In certain of such embodiments, the multi-component mixture (with or without the biologically active agent) is subject to, and has obtained regulatory approval by, a regulatory authority (e.g., the U.S. Food and Drug Administration). In certain embodiments, the biologically active agent is a monoclonal antibody (e.g., adalimumab).

In certain embodiments, the Raman Spectroscopy analysis on the multi-component test mixture is taken from a bioprocess operation (e.g., a filtration or purification operation), either on-line, off-line or at-line. For example, in certain embodiments, the sample could be obtained at regular intervals as part of a Quality Control procedure.

### 4. BRIEF DESCRIPTION OF THE FIGURES

FIG. 1: Raman Spectra of 3 Component (arginine/citric acid/trehalose) buffer system that includes an amino acid, a pH buffer species, and a sugar. This plot was generated using Umetrics SIMCA P+V 12.0.1.0. The X axis is the datapoint number. Each data point is a Raman Shift wavenumber. It could be replotted with Raman Shift wavenumber (cm⁻¹) on the X axis. The data starts with wavenumber 1800 (=Num 0) to 800 (=Num 1000). The Raman spectral raw data is in units of Intensity (related to the number of scattered photons). This Figure shows the mean centered spectral data of the three individual components (in water). The average value of the spectra is 0. The other values are relative to that, probably in standard deviations from the mean.

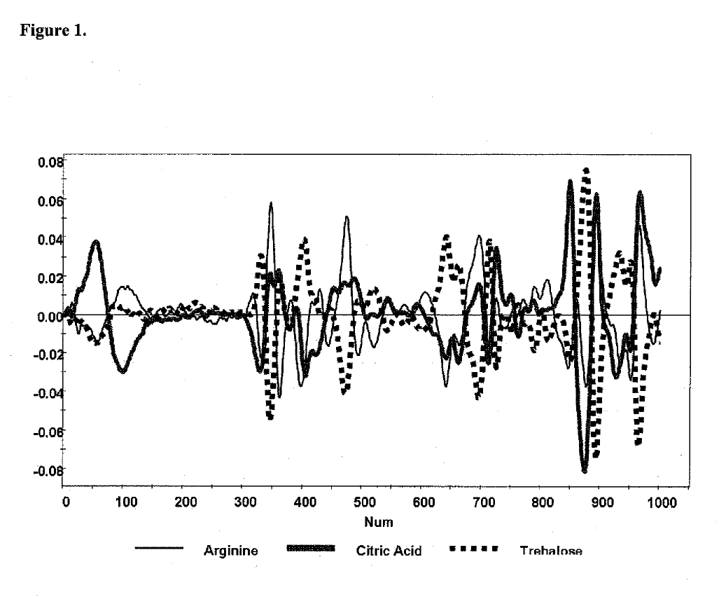

FIG. 2: Comparison of actual vs. predicted concentration for a 3 component buffer system (arginine/citric acid/trehalose) with random values. This Figure was created using the existing model to predict the concentrations of new solutions. The x and y-axis are concentrations (mM).

FIG. 3: Comparison of actual vs. predicted concentration for 3 component buffer system (arginine/citric acid/trehalose) by individual component.

FIG. 4. Pure component raw spectra of 4 component buffer system (mannitol/methionine/histidine/Tween™). The y-axis is spectral intensity, the x-axis is wave number cm⁻¹.

FIG. 5. Pure component raw spectra of 4 component buffer system (mannitol/methionine/histidine/Tween™) The y-axis is spectral intensity, the x-axis is wave number cm⁻¹. FIG. 5 is an more detailed view of the spectra shown in FIG. 4, in which the “fingerprint” region has been expanded.

FIG. 6. Pure component SNV/DYDX/Mean Center spectra of 4 component buffer system (mannitol/methionine/histidine/Tween™). The data shown in FIG. 6 is based on the same data shown in FIGS. 4-5, after all preprocessing: standard normal variate (SNV) for intensity normalization, 1^{st} derivative for base line normalization, and mean centering for scaling.

FIG. 7. Comparison of actual vs. predicted concentration for 4 component buffer system (mannitol/methionine/histidine/Tween™) with random values. This was created using the existing model to predict the concentrations of new solutions.

FIG. 8. Comparison of actual vs. predicted concentration for 3 component buffer system (mannitol/methionine/histidine/Tween™) by individual component.

FIG. 9. Pure component raw spectra for 3 component buffer system with protein (mannitol/methionine/histidine/adalimumab) Raw spectra showing Raman intensity.

FIG. 10. Pure component raw spectra for 3 component buffer system with protein (mannitol/methionine/histidine/adalimumab), with the fingerprint region (800-1700 cm⁻¹) shown in detail.

FIG. 11. Pure component SNV/DYDX/Mean Center—3 component buffer system with protein. The data shown in FIG. 11 is based on the same data shown in FIGS. 9-10, after all preprocessing: standard normal variate (SNV) for intensity normalization, 1^{st} derivative for base line normalization, and mean centering for scaling.

FIG. 12. Comparison of actual vs. predicted concentration for 3 component buffer system with protein by individual component.

FIG. 13. An adalimumab purification process that employs Raman Spectroscopy as part of process and/or quality control.

FIG. 14. On line Raman concentration predictions of a diafiltration process involving a three component mixture of buffer, sugar, and amino acid (methionine/mannitol/histidine).

FIG. 15. Repeated diafiltration process involving a three component mixture of buffer, sugar, and amino acid (methionine/mannitol/histidine). Additional data points included for increased resolution.

FIG. 16. Raman calibration of sugar (mannitol)/protein (adalimumab) solution.

FIG. 17. On line Raman concentration predictions of a diafiltration buffer exchange process where antibody in water is replaced with a mannitol solution to provide a sugar/protein (mannitol/adalimumab) solution. The buffer exchanged is followed by protein concentration.

FIG. 18. Repeat of FIG. 17 experiment where the protein concentration phase is extended to 180 g/L.

FIG. 19. Raman calibration histidine and adalimumab solutions.

FIG. 20. On line Raman concentration predictions of a diafiltration buffer exchange process where protein in water is replaced with a histidine solution. The histidine exchanged is followed by adalimumab concentration.

FIG. 21. Comparison of actual vs. predicted concentration for 2 component buffer system with protein by individual component: Tris concentration; Acetate concentration; and Adalimumab concentration.

FIG. 22. Comparison of actual vs. predicted concentration for 1 component buffer system with protein by individual component: Tween™ concentration; and Adalimumab concentration.

FIG. 23. Conditions of employed when two antibodies (D2E7 and ABT-874) were separately aggregated using photo induced cross-linking of unmodified proteins (PICUP). The antibodies were exposed to the aggregating light source from 0-4 hours.

FIG. 24. Size exclusion chromatographic results of the cross-linking outlined in FIG. 23.

FIG. 25. Raman spectroscopy and the spectra modeled using principal component analysis of D2E7 samples, indicating that aggregated samples have distinct principal component scores and can be discriminated from aggregates using Raman spectroscopy.

FIG. 26. Raman spectroscopy and the spectra modeled using principal component analysis of ABT-874 samples, indicating that aggregated samples have distinct principal component scores and can be discriminated from aggregates using Raman spectroscopy.

FIG. 27. Raman spectroscopy and the spectra modeled using partial least squares analysis of D2E7 samples and ABT-974 samples, indicating some correlation between Raman spectroscopy results and the SEC measurements.

### 5. DETAILED DESCRIPTION

For purposes of clarity and not by way of limitation, the detailed description of the invention is divided into the following subsections:

(i) Definitions

(ii) Applicable Processes and Systems; and

(iii) Raman Spectroscopy Apparatuses and Techniques.

### 5.1 Definitions

As used herein, the term “saccharide” includes compounds of the general formula (CH₂O)ₙ and derivatives thereof, and further includes monosaccharides, disaccharides, trisaccharides, polysaccharides, sugar alcohols, reducing sugars, nonreducing sugars, etc. Non-limiting examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc.

As used herein, the term “surfactant” refers to a surface-active agent. In one embodiment, the surfactant is a nonionic a surface-active agent. Examples of surfactants include, but are not limited to, polysorbate (for example, polysorbate 20 and, polysorbate 80); poloxamer (e.g., poloxamer 188); Triton™; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics™, PF68™ etc); and the like.

As used herein, the term “pH buffer” refers to a buffered solution that resists changes in pH by the action of its acid-base conjugate components. Examples of pH buffers that will control the pH include tris, trolamine, phosphate, bis-tris propane, histidine, acetate, succinate, succinate, gluconate, histidine, citrate, glycylglycine and other organic acid buffers.

As used herein, “biologics” refers to cells, molecules, organelles (natural or synthesized) or other matter derived from a living organism of non-synthetic chemical origin, either from recombinant or natural sources. Examples include, but not limited to, DNA, RNA, virus, virus sub units, virus like particles, peptides (synthetic and natural), proteins. Any of these molecules can provide Raman signal that can be measured and used in monitoring and control of systems.

As used herein, the term “provided in an industrial scale” refers to a bioprocess in which, for example, a therapeutic (e.g., a monoclonal antibody for administration to a human) or other end product is produced on a continuous basis (with the exception of necessary outages for maintenance or upgrades) over an extended period of time (e.g., over at least a week, or a month, or a year) with the expectation of generating revenues from the sale or distribution of the therapeutic or other end product of commercial interest. Production in an industrial scale is distinguished from laboratory “bench-top” settings which are typically maintained only for the limited period of the experiment or investigation, and are conducted for research purposes and not with the expectation of generating revenue from the sale or distribution of the end product produced thereby.

### 5.2 Applicable Processes and Systems

Certain embodiments of the present application employ Raman spectroscopy techniques to characterize components (e.g., multi-component mixtures) used in bioprocess operations. For example, in certain embodiments, Raman spectroscopy can be used to characterize formulations that are intended to be combined with a biologically active agent (e.g., a monoclonal antibody). These formulations, sometimes referred to as “formulation buffers” are typically multi-component mixtures that determine excipient levels in biologics. For example, such formulations generally include one or more of the following: a pH buffer (e.g., a citrate, Tris, acetate, or histidine compound), a surfactant (e.g., polysorbate 80), a sugar or sugar alcohol (e.g., mannitol) and/or an amino acid (e.g., L-arginine or methionine). Errors in formulation buffers often result in rejected batches, which in turn result in significant loses.

In certain embodiments, Raman spectroscopy techniques can be used to identify protein aggregations. For example, but not by way of limitation, the Raman spectroscopy techniques of the present invention can, in certain embodiments, identify aggregations of protein Drug Substance and Drug Product samples, such as antibody Drug Substance and Drug Product samples.

In certain embodiments, Raman spectroscopy techniques can be used to verify excipient concentrations in Drug Substance and Drug Product samples. In certain of such embodiments, excipients concentrations are verified as part of a quality control process based on a single reading, obviating the need for a series of analytical tests. In certain embodiments, Raman spectroscopy can also be used in bioprocesses involving product dilutions and pH adjustments.

In certain embodiments, Raman spectroscopy can be used to test and characterize formulations present in filtration operations (e.g., ultrafiltration/diafiltration processes), such as filtration operations in which a biologically active agent, such as a monoclonal antibody (e.g., adalimumab) is purified. For example, but not by way of limitation, the Raman spectroscopy techniques of the present invention can be used to obtain samples obtained on-line or off-line to ascertain both the identity and quantity of the components present in a single reading. In certain embodiments, protein concentrations can be determined in addition to excipient concentrations. In certain of such embodiments, protein concentrations in the range of 0 to 150 mg/ml can be analyzed.

In certain embodiments, Raman spectroscopy can be used to monitor, verify, test and hence control bioprocess operations. The unit operations that are used with bioprocess operations, e.g., chromatography, filtration, pH changes, composition changes by addition of components or dilution of solutions, all result in mixtures composed of organic or inorganic components and biological molecules. Accordingly, measuring rapidly and accurately the composition of intermediates, for example, by employing Raman spectroscopy, provides opportunities to improve and maintain consistency and quality of the operations as well as the biological product.

In certain embodiments, the measurement of the composition of individual components in a mixture by Raman spectroscopy allows for accurate preparation of such mixtures, with and without the presence of the biologic molecule. For example, in certain embodiments, such a measurement will be useful in preparation of buffer solutions used extensively in bioprocess operations with benefits of improving consistency of the preparation or providing near real time preparation of the buffer solutions. In certain embodiments, this will eliminate the need for elaborate equipment for preparation, holding and delivery of buffer solutions. In certain embodiments, the use of Raman spectroscopy allows for the testing and release of buffer solutions can be provided in which potential errors in the buffer formulations (e.g., chemical component concentrations, wrong chemicals, etc.) are detected in real-time with simple instrumentation. Formulations that can be tested include, but are not limited to, protein-free three-component formulations (buffer+sugar+amino acid), protein and sugar formulations, protein and surfactant formulations, and protein and buffer formulations.

In certain embodiments, accurate measurement of solution composition allows for adjustment of biological solutions so that the right target composition of additives (anion, cation, hydrophobic, solvents, etc.) can be achieved. Currently such measurements are tedious and require sophisticated analytical methods that are not amenable to implementation to real time use. The use of Raman spectroscopy allows for measurements that provide a very high degree of assurance with documentation, which is an expectation in regulated industries.

In certain embodiments, the techniques of the instant invention allow for the ability to monitor and control protein—protein reactions, protein—small molecule reactions, and/or protein modifications that are achieved by chemical, physical or biological means. In certain of such embodiments, the unique biochemical signature of the reactant (biologic in its original state) and the product (biologic in its final state), as well as other reactants/catalysts that are either chemical or biological in nature are monitored using Raman spectroscopy. Monitoring the reactant(s) and product(s) in this fashion allows for, among other things, feed back control of reaction conditions and reactant amounts. It is also possible, in certain embodiments, to design a system to remove reaction by products and/or products continually to optimize, improve or maintain product quality or performance of such systems.

In certain embodiments, Raman spectroscopy also allows for biologic product isolation and purification in chromatography operations. In certain of such embodiments, the elution of product/product variants/product isoforms or impurities can be monitored and fractionation of column effluent can be performed based on desired product quality or process performance. In certain embodiments, it is also possible to apply Raman spectroscopy to the isolation/enrichment of fractions in other unit operations, such as, but not limited to, filtration and non-chromatographic separations.

In certain embodiments, Raman spectroscopy is capable of being deployed as a non-invasive tool. For example, but not by way of limitation, Raman spectroscopy measurements can be made through materials that do not interfere with the signal. This provides additional unique advantages in bioprocess operations where maintaining the integrity of the containers/vessels containing these mixtures is critical.

In certain embodiments, Raman spectroscopy can be an extremely valuable means of detecting “contamination” of a solution with other components. In certain of such embodiments, Raman spectroscopy data obtained from a contaminated solution is compared with the expected spectra using statistical or spectral comparison techniques and, if different, can allow for the rapid detection of errors in formulation of these solutions, before they are used in bioprocesses.

In certain embodiments, as demonstrated through an example below as a proof of concept, concentration of antibody in a mixture containing impurities from the cell culture harvest materials including host cell proteins, DNA, lipids etc can be measured quantitatively using Raman Spectroscopy. In such embodiments, the said method can be used to monitor influents and effluents from bioprocess operations containing unpurified mixtures. Examples could include, but not limited to loading and elution operations for columns, filters, and non-chromatographic separation devices (expanded bed, fluidized bed, two phase extractions etc). The example provided demonstrates that the antibody concentration from 0.1 to 1 g/L can be quantified in a matrix that comprises the unbound fraction from a protein A affinity chromatography column that was loaded with a clarified harvest solution prepared from a chemically defined media based cell culture process. If Raman spectroscopy is incorporated in-line, then such a measurement will enable direct monitoring and control of the column loading, enabling consistent and optimal loading of the columns either at a predefined binding capacity that represents either a percent of the dynamic binding capacity or static (equilibrium) capacity. It is obvious to one skilled in the art to apply such technology to various other operations as mentioned above.

In certain embodiments, Raman spectroscopy can be used for quality control and/or feedback control in bioprocess purification operations (e.g., to control in-line buffer dilution for an adalimumab purification process). In certain of such embodiments, Raman spectroscopy can be used for quality control and/or feedback control in processes involving protein conjugation reactions or other chemical reactions (e.g., a liquid-phase Heck reaction), as described in Anal. Chem., 77:1228-1236 (2005), hereby incorporated by reference in its entirety.

In various embodiments of the presently-disclosed subject matter, the Raman spectroscopy techniques disclosed herein are employed in bioprocess operations that are provided in an industrial scale, as defined above.

Although, solely for the sake of convenience, the subject matter of the present application is described largely in the context of bioprocess methods, systems for conducting the bioprocesses themselves are also provided (see, e.g., Example 13). Accordingly, certain embodiments of the present application provide systems for conducting bioprocess operations, including bioprocess systems provided in an industrial scale, in which Raman probes are in fluid communication with samples taken on-line or off-line from the respective process. Information regarding the systems themselves can be obtained from the description of the corresponding process.

### 5.3 Raman Spectroscopy Apparatuses and Techniques

Raman spectroscopy is based on the principle that monochromatic incident radiation on materials will be reflected, absorbed or scattered in a specific manner, which is dependent upon the particular molecule or protein which receives the radiation. While a majority of the energy is scattered at the same wavelength (Rayleigh scatter), a small amount (e.g., 10⁻⁷) of radiation is scattered at some different wavelength (Stokes and Antistokes scatter). This scatter is associated with rotational, vibrational and electronic level transitions. The change in wavelength of the scattered photon provides chemical and structural information.

In certain embodiments, Raman spectroscopy can be performed on multi-component mixtures to provide a highly specific “fingerprint” of the components. The spectral fingerprint resulting from a Raman spectroscopy analysis of a mixture will be the superposition of each individual component. The relative intensities of the bands correlate with the relative concentrations of the particular components. Accordingly, in certain embodiments, Raman spectroscopy can be used to qualitatively and quantitatively characterize a mixture of components.

Raman spectroscopy can be used to characterize most samples, including solids, liquids, slurries, gels, films, powders and some gases, with a very short signal acquisition time. Generally, samples can be taken directly from the bioprocess at issue, without the need for special preparation techniques. Also, incident and scattered light can be transmitted over long distances allowing remote monitoring. Furthermore, since water provides only a weak Raman scatter, aqueous samples can be characterized without significant interference from the water.

The applicable processes and compositions described herein can be analyzed based on commercially available Raman spectroscopy analyzers. For example, a RamanRX2™ analyzer, or other analyzers commercially available from Kaiser Optical Systems, Inc. (Ann Arbor, Mich.) can be employed. Alternatively, Raman analyzers commercially available from, for example, PerkinElmer (Waltham, Mass.), Renishaw (Gloucestershire, UK) and Princeton Instruments (Trenton, N.J.). Technical details and operating parameters for the commercially available Raman spectroscopy analyzers can be obtained from the respective vendors.

Suitable exposure times, sample sizes and sampling frequencies can be determined based on, for example, the Raman spectroscopy analyzer and the process for which it is employed (e.g., in processes providing real-time monitoring of UF/DF bioprocess operations). Similarly, proper probe placement can also be determined based on the analyzer and process for which the analyzer is employed. For example, the sample size for the immersion probe to provide an adequate signal can be less than 20 mL, or less than 10 mL (e.g., 8 mL or less). The exposure time to provide an adequate signal can be less than 2 minutes, or less than 1 minute (e.g., 30 seconds).

For example, for components for which quantization is desired, and that exist at more than one pH dependent ionization forms (e.g., histidine), raman calibrations can be conducted at varying concentrations, and/or at various pH's to predict the concentration over a given pH range, such that measurement of the component (e.g., histidine) is not pH-dependent. For example, calibration models for histidine in different pH-dependent forms can be used to measure and quantify histidine in various ionized forms such that solution properties can be ascertained. Signal processing can be performed, which can include an intensity correction (e.g., standard normal variate (SNV)) and/or baseline correction (e.g., a first derivative).

Exposure times can be determined by measuring CCD saturation of representative test solutions and ensuring that they are within the acceptable instrument range (e.g., 40-80%). As noted, above, in some embodiments, pH control or pH range modeling is employed for particular components (e.g., buffers such as histidine). In some embodiments, incident light is minimized, which can be achieved, for example, by use of a cover to block ambient light sources from interfering with the spectroscopy (e.g., aluminum foil).

In certain embodiments, in which, for example, a protein (such as an antibody) is concentrated with non-charged species, the protein occupies a significant volume of the solution, excluding a significant amount of solute. This results in an net decrease in the concentration of the non-charged species. This effect is referred to as “Volume exclusion,” which is proportional to the protein concentration.

In certain embodiments, such as those embodiments involving assays of charged components, a Donnan Effect occurs because at higher concentrations, protein charge becomes a significant contribution to the overall charged species in solution. Since an equilibrium is expected to be established on either side of the membrane, the electroneutrality requirement results in a net decrease in positively charged species (e.g., buffer species) on the retentate side of the membrane. This phenomenon is called the Donnan effect.

According to certain embodiments of the present application, a RamanRX2™ analyzer is employed. This analyzer, as well as other commercially available Raman analyzers, provides the capability of monitoring up to four channels with simultaneous full-spectral coverage. In certain embodiments, standard NIR laser excitation is employed to maximize sample compatibility. Programmable sequential monitoring formats can be employed, for example, by the RamanRX2™ analyzer, and the apparatus is compatible with process optics, enabling one analyzer type to be employed from the discovery phase to the production phase. A portable enclosure and fiber optic sampling interface allows the analyzer to be used in multiple locations.

In certain embodiments of the presently disclosed subject matter, at least one multi-component mixture standard containing pre-determined amounts of known components (i.e., multi-component mixture standards) are characterized by Raman spectroscopy in order to obtain a model for use with mixtures with unknown components and/or unknown concentrations of known or unknown components (e.g., a calibration curve). Preferably, a series of multi-component mixture standards with pre-determined amounts of known components are characterized via Raman spectroscopy for purposes of obtaining a model.

Methodologies for obtaining a model for use with mixtures with unknown components and/or unknown concentrations of known or unknown components can be determined by persons of ordinary skill in the art. For example, a Partial Least Squares Regression Analysis based on the principal components that are expected to be present in multi-component test mixtures. Also, software programs available from Raman spectroscopy vendors can be employed to design multi-component mixture standards, which in turn can be used to develop the model for use with the multi-component test mixtures.

Furthermore, it is understood that reference to “providing a multi-component mixture standard with pre-determined amounts of known components” and “performing a Raman Spectroscopy analysis on the multi-component mixture standard,” and more generally, developing a model to characterize multi-component mixtures with unknown components or unknown concentrations of components includes both parallel analysis (i.e., data obtained “on-site”), as well as reference to previously obtained or previously recorded results (e.g., Raman spectra fingerprints) for multi-component mixture standards, i.e., multi-component mixtures with known components with known concentrations. For example, reference to Raman spectra results obtained from vendor product literature in encompassed by “providing a multi-component mixture standard with pre-determined amounts of known components” and “performing a Raman Spectroscopy analysis on the multi-component mixture standard.”

### 6. EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### 6.1 Testing of 3-Component Formulation Buffers

Formulation buffers containing predetermined mixtures of arginine, citric acid, and trehalose were prepared with a water solvent. Components were varied from 0 to 100 mM.

Raman spectra over the range of 800 to 1700 cm⁻¹ were obtained for 15 mL aliquots of each mixture using a RAMANRXN2™ Analyzer (2 spectra/mixture). The spectral filtering parameters were set to a standard normal variance (SNV) intensity normalization, a 1st derivative (gap) baseline correction with 15 point smoothing, and mean centering difference spectra with the average intensity value=0. This is considered to be a data scaling rather than a spectral filter. The spectra were collected using an immersion probe with an exposure time of 30 seconds per sample.

Principal Components Methodology was used to develop a model. A PLS (Partial Least Squares projections to latent structures) model was applied to each of the three components to determine inter-component correlations. This result is a linear model that translates spectral intensity (e.g. from 1700-800 cm-1) to concentration (ax1+bx2+ . . . +zx900=concentration). The software used for the calibration results shown here was GRAMS/AI V 7.02 with the PLSplus/IQ add-in from Thermo Galactic. SIMCA P+ was used for many of the graphs and experimental model creation. The samples were cross validated by removing two samples. Data analysis was conducted so that the steps of testing for correlations and cross-validating were iterated until the inter-component correlations were below an error threshold of 2%. Accurate quantization of buffer components (e.g., within 2%) can be provided with a single reading.

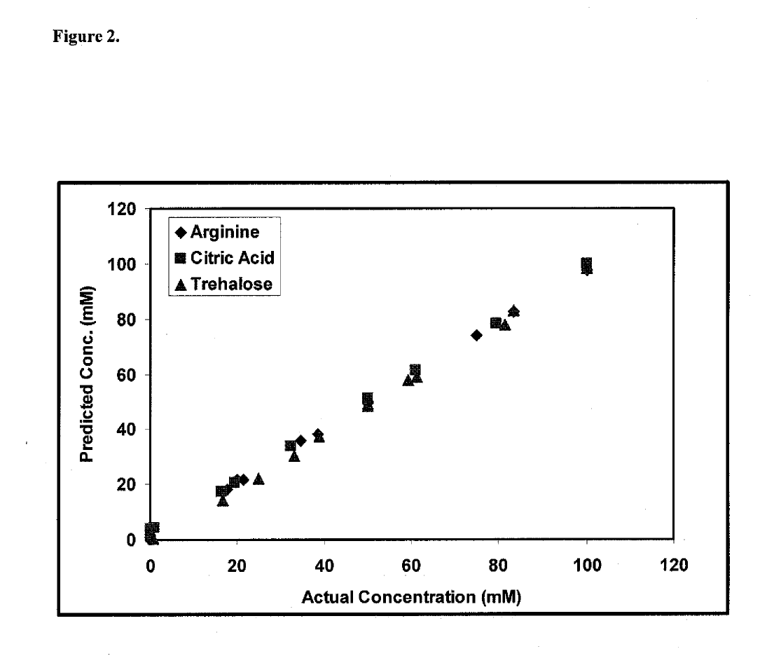

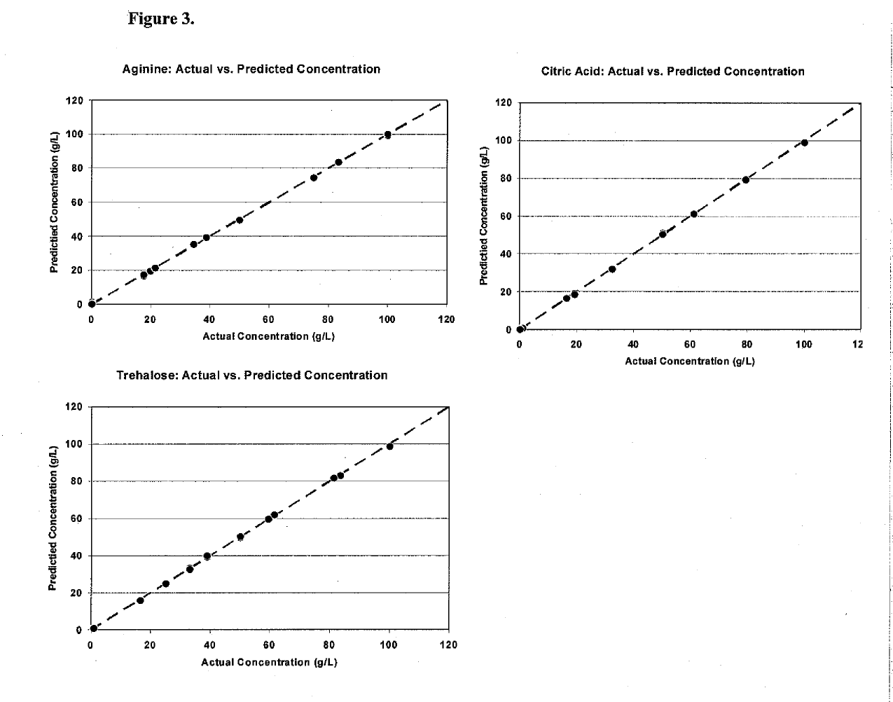

Calibration curves can be obtained using Random Mixture Design. The 3-component model developed above was used to generate predictions about spectra of random mixtures of arginine, citric acid, and trehalose. These predictions were compared against the actual spectra to confirm that the model is with the pro-determined tolerance limit of ±2%. The results are shown in FIGS. 2 and 3. Independent measurements were obtained of random mixtures to verify that the model can be used for making measurements.

### 6.2 Testing of 4-Component Formulation Buffers

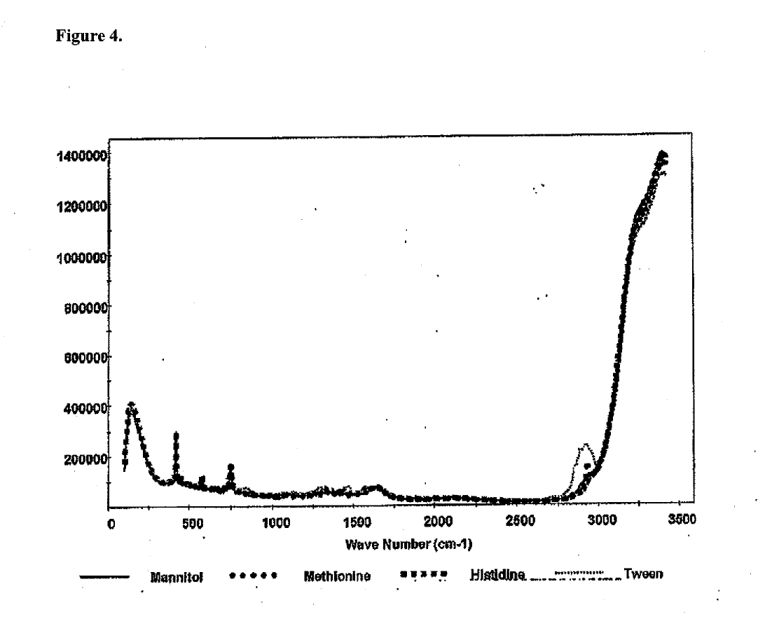

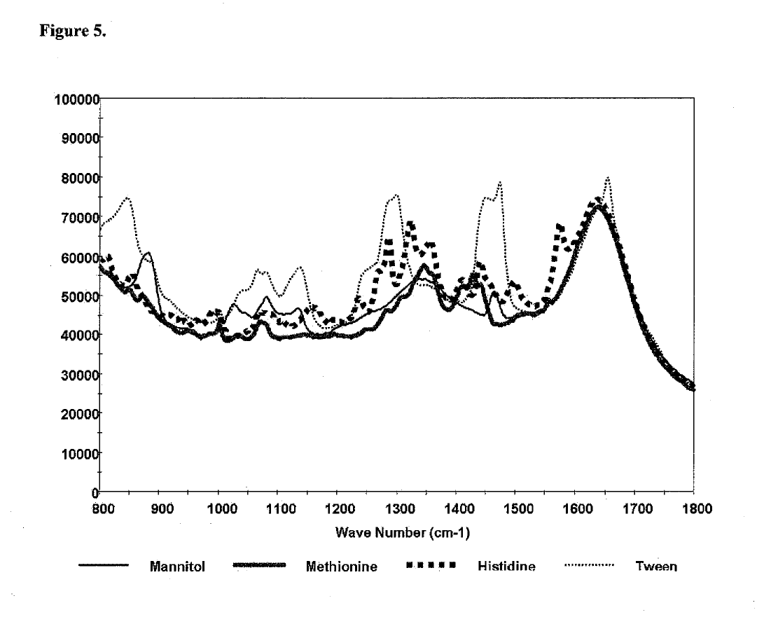

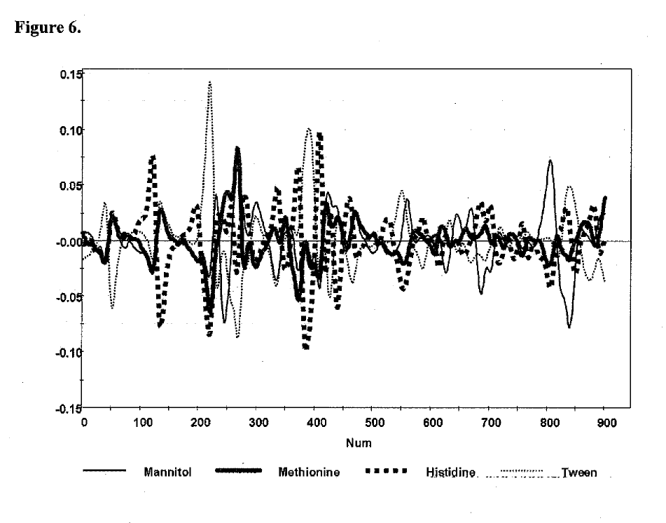

The methodology of Example 6.1 was applied to formulation buffers containing 4 components, wherein the components were mannitol, methionine, histidine, and Tween™ (polysorbate 80). The measured spectra of the predetermined mixtures are shown in FIG. 4-6. The wave numbers range from the Far-IR region to the Mid-IR region. Due to limitations with the sapphire cover, the range from 100-800 cm⁻¹ can be disregarded in this particular example, and calibration occurs from 800-1800 cm⁻¹.

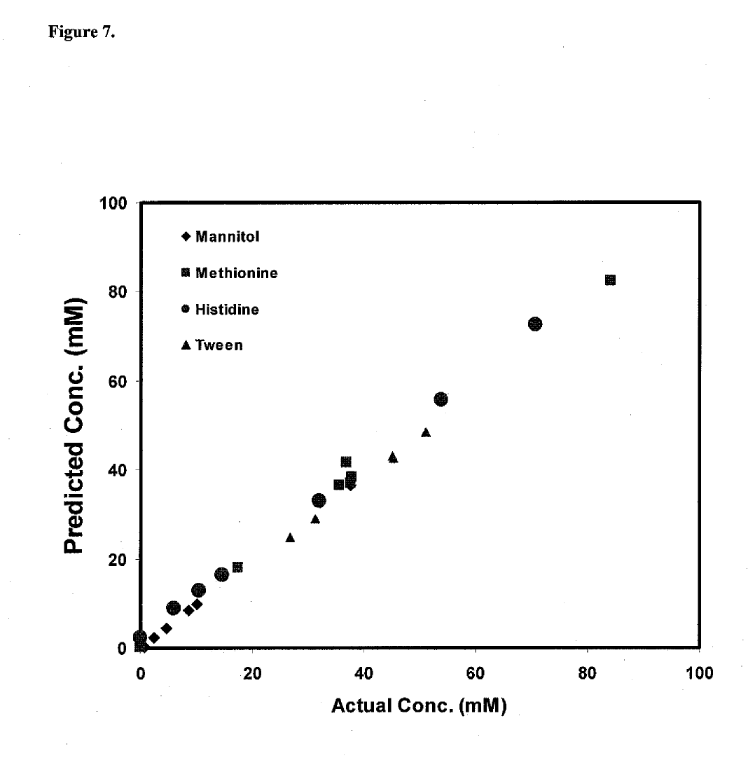

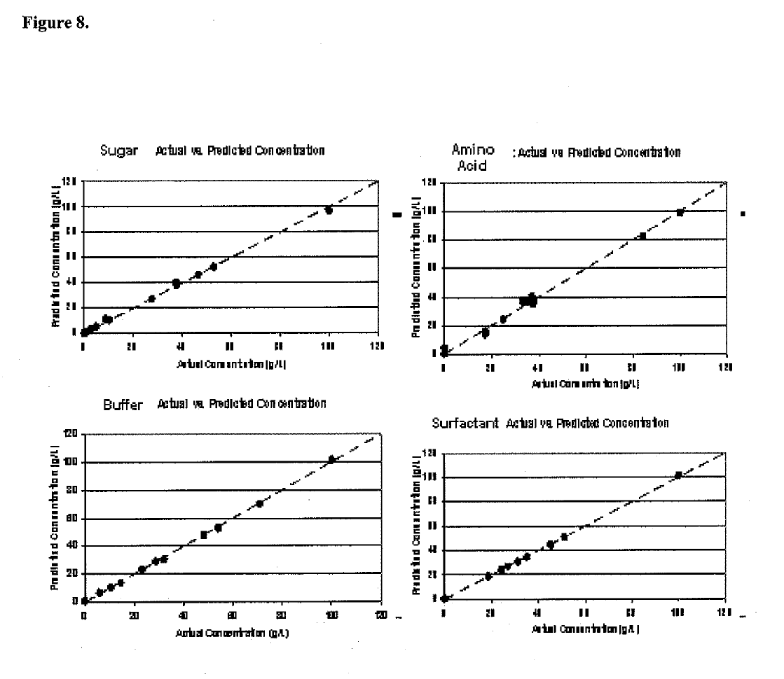

A model was obtained for a 4 component buffer system in the same manner as the 3 component model obtained in Example 6.1. The predictions based on the model obtained were compared against the actual spectra of random mixtures to confirm that the model is sufficiently accurate. The results are shown in FIGS. 7 and 8.

### 6.3 Testing of 3-Component Formulation Buffers with Protein

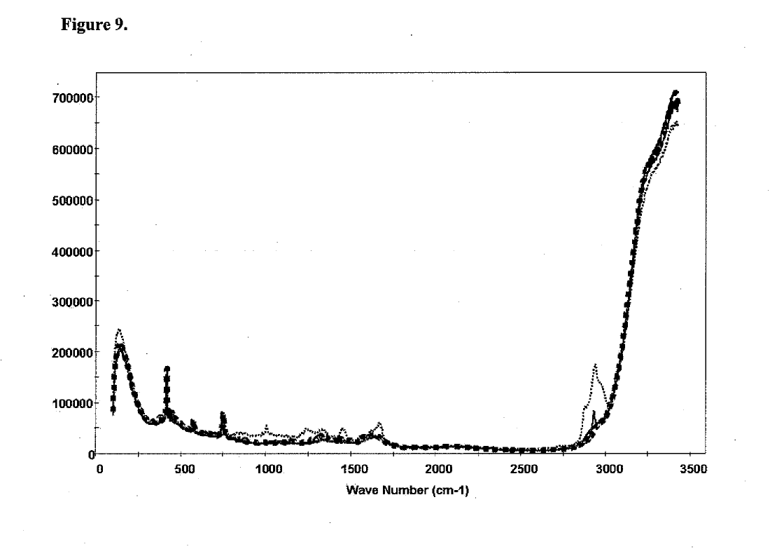

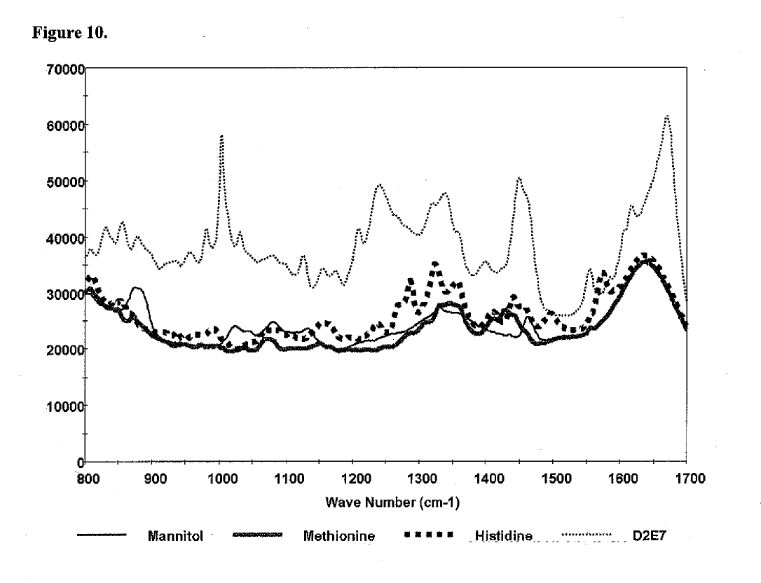

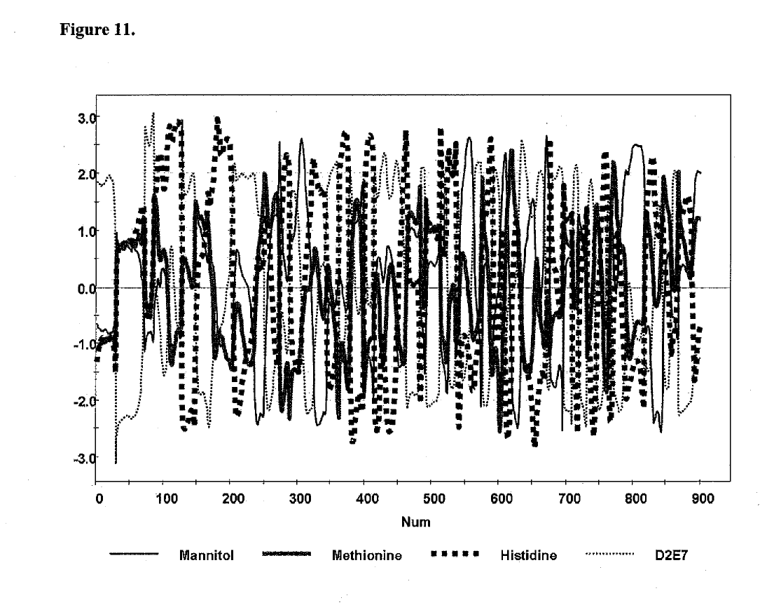

The methodology of Example 6.2 was applied to formulation buffers containing 3 components along with a protein at a concentration in the range of 0 to 100 mg/ml. The components were mannitol, methionine, histidine, and D2E7 (adalimumab). The measured spectra of the predetermined mixtures are shown in FIGS. 9-11.

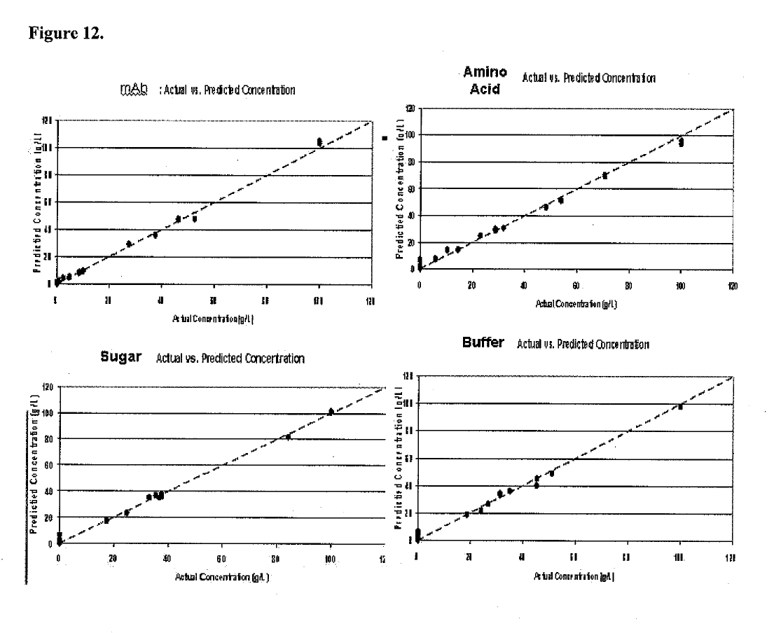

A model was obtained for a 3 component buffer system with protein in the same manner as the 4 component model obtained in Example 6.2. The predictions based on the model obtained were compared against the actual spectra of random mixtures to confirm that the model is sufficiently accurate. The results are shown in FIG. 12. The coefficient of determination (R²) and standard error of cross-validation (SECV) values of the actual versus predicted spectra are show in Table 1 below.

TABLE 1
Model Fit Summary

| Component | R² | SECV (g/L) |
|---|---|---|
| Adalimumab | 0.995 | 1.96 |
| Mannitol | 0.994 | 2.35 |
| Methionine | 0.989 | 3.27 |
| Histidine | 0.992 | 2.75 |

### 6.4 Adalimumab UF/DF Process

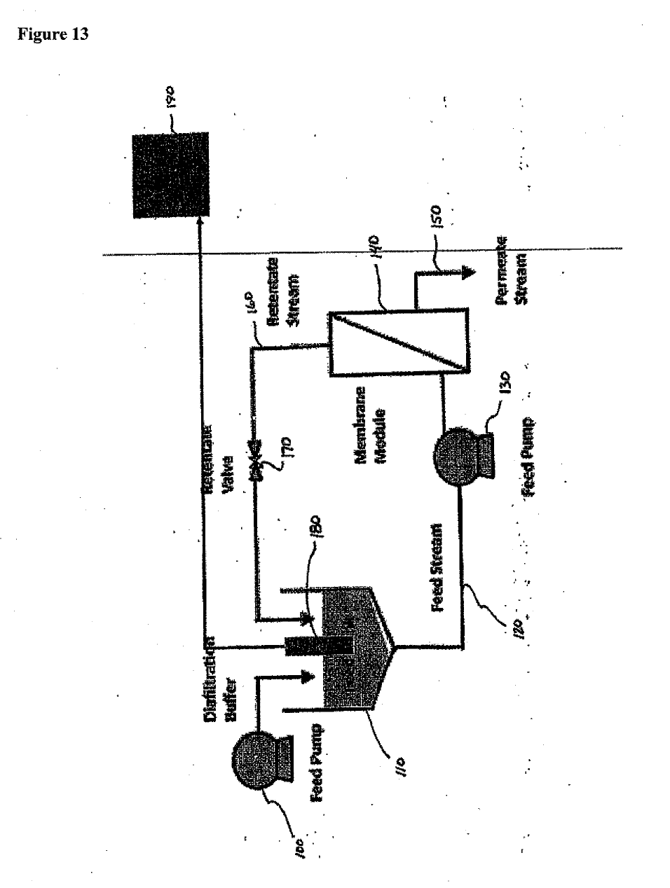

An ultrafiltration/diafiltration process (UF/DF) is established to introduce excipients into a solution of adalimumab, shown in FIG. 13. A feed pump (100) provides cross flow across the tangential flow filtration membrane, passing the adalimumab containing solution in the reservoir over the membrane. The diafiltration buffer (formulation buffer, containing Methionine, Mannitol and Histidine) is pumped into the reservoir to match the filtration rate of the membrane (liquid flowing through the permeate side of the membrane) (110). A feed stream (120) exiting the feed tank is directed by a pump (130) to a membrane module (140). A permeate stream (150) containing water, buffer components, and the like having a relatively smaller molecular size passes through the membrane module. A retentate stream (160) containing concentrated adalimumab is directed back to the feed tank, as controlled by a retentate valve (170).

A Raman probe (180), compatible with a RamanRX2™ analyzer (190) from Kaiser Opticals is placed within the feed tank to provide the ability to characterize the content of the tank periodically. The spectra obtained will be converted to component concentrations using the calibration file and hence the progress of the diafiltration process can be monitored. In addition, the changes in excipient concentrations that happen due to increase in concentration of the protein (caused by Donnan and charge exclusion effects) can be monitored and optionally controlled. Other Raman systems, besides a RamanRX2™ analyzer could also be used to characterize online samples from the ultrafiltration/diafiltration process on a regular basis as part of the Quality Control of the adalimumab purification process. For example, the results from the Raman analysis can be used to assess the completion of the diafiltration process and the final excipient concentrations.

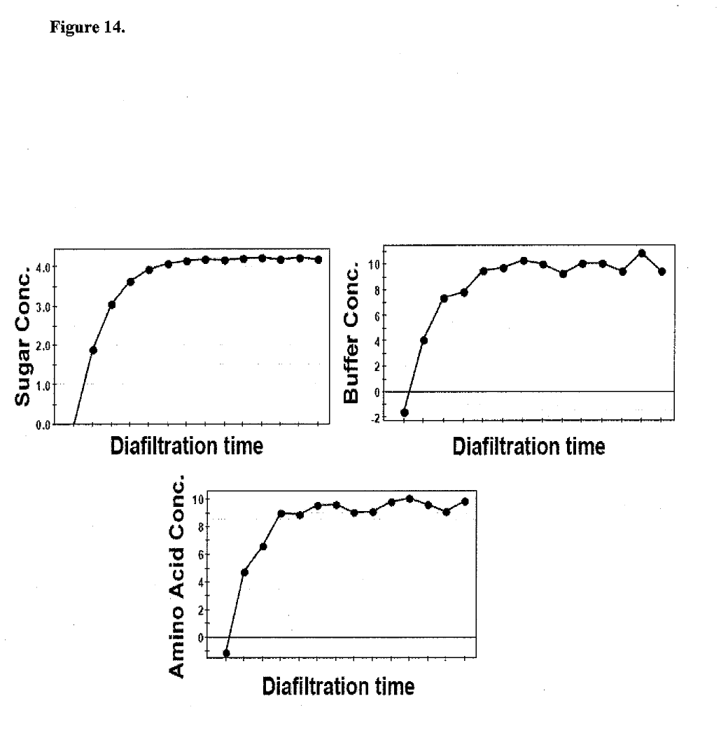

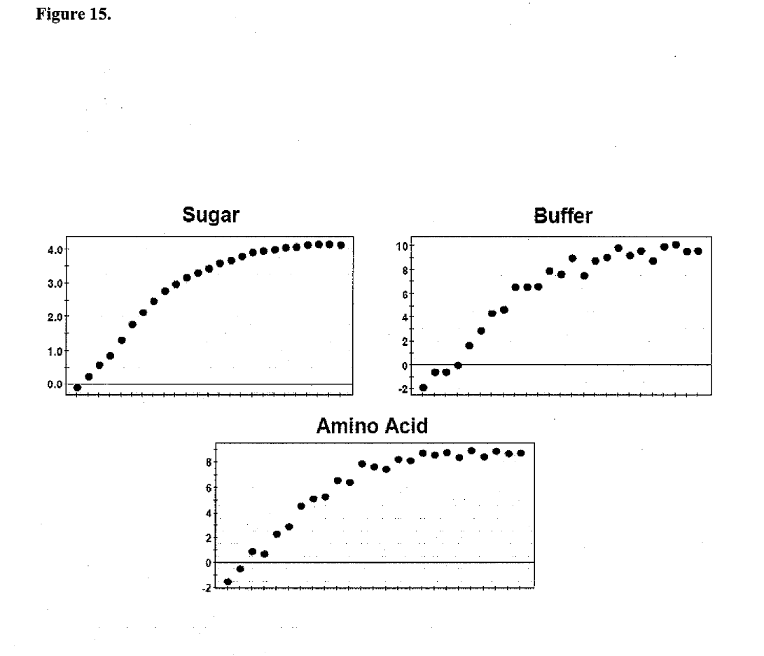

A mixture of histidine, mannitol and methionine were diafiltered across a UF/DF membrane. The raman probe was placed in the retentate reservoir. Raman Spectra were obtained at specified intervals, with each reading consisting a 30 sec exposure, repeated 10 times (10 scans). FIGS. 14-15 show the change in concentration during diafiltration. As expected the concentration of individual components increase during diafiltration reaching a plateau.

FIGS. 14-15 provide results from the on-line monitoring of the diafiltration process. In FIG. 14 sugar, buffer and amino acid concentrations are provided for various diafiltration times. As shown in FIGS. 14 and 15, amino acid is methionine, and concentration (mM) is plotted on the y-axis, sugar is mannitol, and w/v % is plotted on the y-axis, and buffer is histidine, and concentration (mM) is plotted along the y-axis. The x-axis for each of the plots in FIGS. 14-15 is retention time, in which concentrations from 0 to 81 minutes were measured and plotted along the x-axis.

Next, adalimumab at approximately 40 mg/ml present in water was diafiltered into a sugar solution over 7 diavolumes across a 5 kiloDalton UF/DF membrane (0.1 sq. m). The raman probe was placed in the retentate reservoir. Raman Spectra were obtained at specified intervals, with each reading consisting of a 30 second exposure time, repeated 10 times (10 scans). Subsequently the protein was concentrated to 140 g/L.

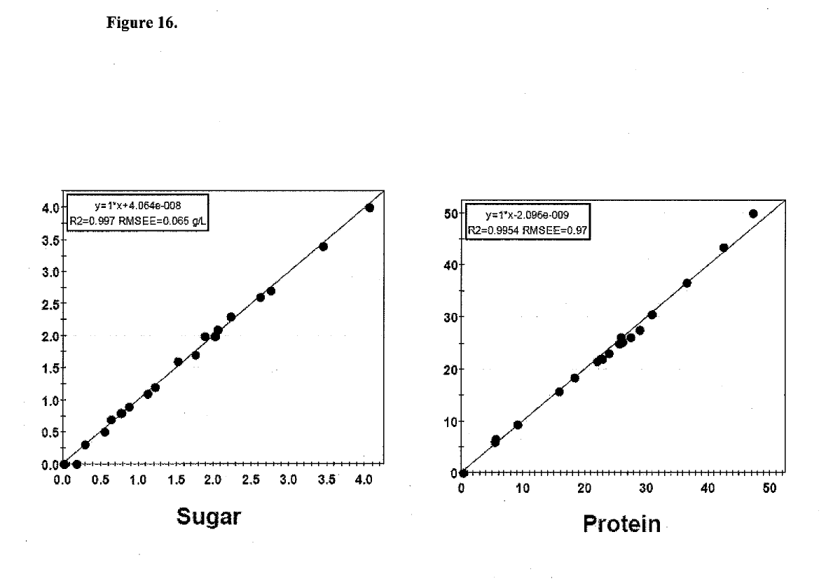

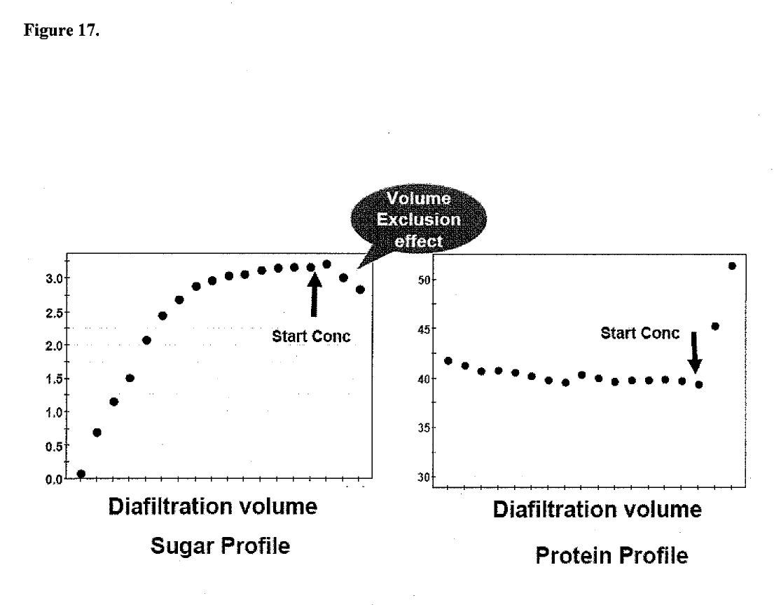

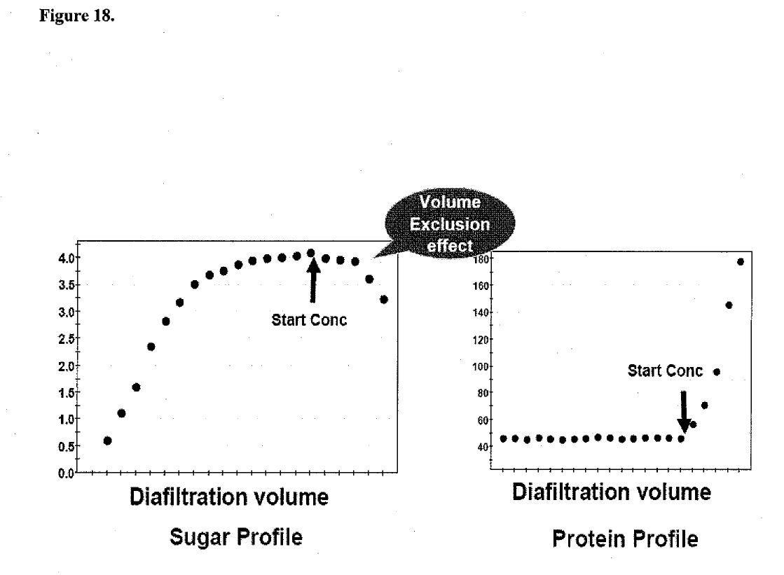

FIG. 16 provides calibration data obtained from the sugar/protein system (mannitol/adalimumab) that is employed in a UF/DF system and measured as described above. The calibration curve from FIG. 16 was used to ascertain mannitol and adalimumab concentrations in FIGS. 17 and 18. FIGS. 17 and 18 show the change in concentration during diafiltration of the sugar. The plot on the right shows the protein concentration during diafiltration and then subsequent ultrafiltration. In FIGS. 17 and 18, sugar concentration (%) is plotted versus retention volumes (from zero to 6), and adalimumab concentration (g/l) is plotted versus retention volumes (from zero to 6).

As expected the concentration of sugar increase during diafiltration reaching a plateau. The protein reaches the target concentration. In FIG. 17, a model calibrated to 50 g/L was used. FIG. 18 shows the sugar and protein concentrations calculated using calibrations obtained with 120 g/L protein and sugar mixtures.

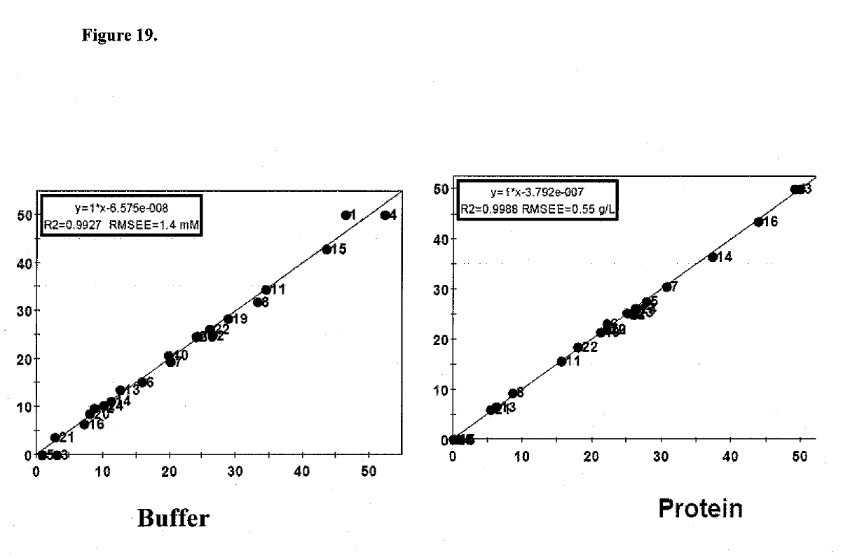

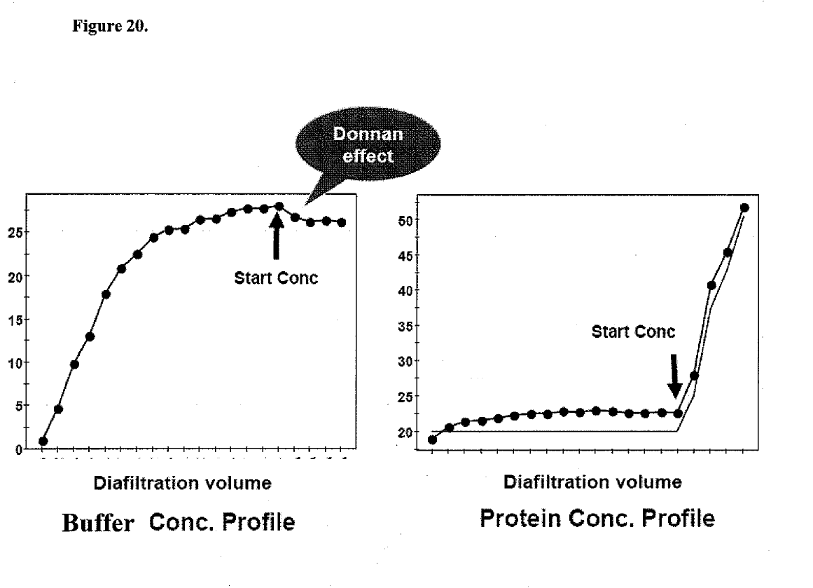

Adalimumab at approximately 20 mg/ml present in water was diafiltered into a histidine solution (50 mM) over 7 diavolumes across a 5 kiloDalton UF/DF membrane (0.1 sq. m). The raman probe was placed in the retentate reservoir. Raman Spectra were obtained at specified intervals, with each reading consisting a 30 sec exposure, repeated 10 times (10 scans). Subsequently the protein was concentrated to 50 g/L. FIG. 19 provides calibration data obtained from the buffer (histidine)/protein (adalimumab) system. This is the calibration model for histidine/adalimumab mixture for up to 50 g/L protein. FIG. 20 provides a plot of diafiltration volumes (from 0 to 6 diafiltration volumes) versus histidine concentration (nM) and adalimumab concentrations (g/l) for low concentrations of buffer and protein in a buffer/protein system.

The plots show the change in concentration during diafiltration of the histidine (nM). The plot on the right shows the protein concentration (g/l) during diafiltration and then subsequent ultrafiltration. As expected the concentration of sugar increase during diafiltration reaching a plateau. The protein reaches the target concentration. In this plot (FIG. 19), a model calibrated to 50 g/L was used. The concentration in the plot is lower than expected, due to the model limitation, which was later identified to be related to the ionization of histidine. Models can correlate the ionized state of histidine to the actual total histidine concentration and solution properties.

The data demonstrates the capability to monitor low and high concentration UF/DF operations with a protein and an additional single component. Concentrations can be read every 3 minutes thus providing the ability to monitor concentrations in real time (or near real-time). In the sugar/protein system, very high accuracy was obtained with sugar for all concentrations of protein. In the buffer/protein system, high buffer accuracy was obtained at higher buffer concentrations and lower protein concentrations. The ability to detect and measure volume exclusion effects and Donnan effects is also provided in real-time (or near real-time). Thus Raman spectroscopy is useful as a tool for excipient concentration measurements in protein solutions, and also provides the ability to measure protein concentrations in addition to excipient concentrations to provide process control.

### 6.5 Testing of 2-Component Formulation Buffers with Protein

The methodology of Example 6.1 was applied to formulation buffers containing 2 components, Tris and Acetate, and a protein, Adalimumab. The components were included in the following ranges: Tris 50-160 mM; Acetate 30-130 mM; and Adalimumab 4-15 g/L.

Calibration curves can be obtained as outlined in Example 6.1. The models developed above were used to generate predictions about spectra of mixtures of Tris, Acetate and Adalimumab, in samples prepared according to the concentrations of Table 2:

TABLE 2

| Tris (mM) | Acetate (mM) | Ab (g/L) |
|---|---|---|
| 160 | 30 | 4.0 |
| 50 | 130 | 4.0 |
| 50 | 30 | 15.0 |
| 50 | 93 | 8.1 |
| 85 | 30 | 11.5 |
| 99 | 85 | 4.0 |
| 105 | 80 | 9.5 |
| 106 | 59 | 6.2 |
| 100 | 63 | 6.4 |
| 53 | 36 | 14.0 |
| 80 | 72 | 7.4 |
| 102 | 51 | 7.5 |
| 52 | 63 | 11.2 |
| 128 | 52 | 4.8 |
| 128 | 37 | 6.4 |

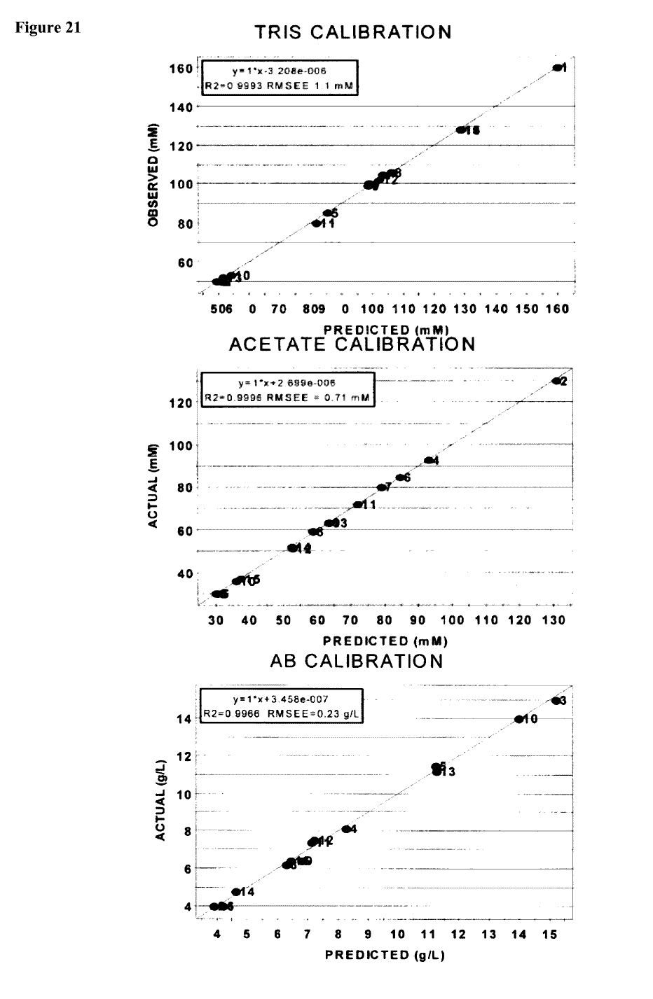

These predictions were compared against the actual spectra to confirm that the model falls within predetermined tolerances. The results are shown in FIG. 21A-C.

### 6.6 Testing of Cell Culture Harvest with Protein

The methodology of Example 6.1 was applied to formulation buffers containing 1 component, Tween™, and a protein, Adalimumab. The cell culture media was harvested from a cell culture batch, filtered, and loaded onto a protein A column. The protein A column flow through was pooled and then sterile filtered prior to storage and testing.

This methodology would be used to determine the end point of a protein A column load. Filtered cell culture harvest would be applied to a capture column (typically protein A). The current method for monitoring column load output uses A280 absorbance. The culture harvest, however, contains many constituents that absorb light at 280 nm. The A280 absorbance is usually saturated, rendering the A280 method incapable of measuring antibody breakthrough during the column load phase.

The Raman spectrometer offers a specific measurement for antibody in a capture column load output stream (the column flow-through). This test simulates a proposed on-line antibody measurement by spiking various concentrations of purified antibody API drug substance (e.g., Adalimumab) into a pool of protein A flow-through. The API sample used for the spiking experiments contained 0.1% Tween™. During a direct spiking experiment, the Tween™ concentration would change in direct proportion with the antibody, and could be mistaken for antibody during the Raman spectral calibration. To avoid this, the Tween™ was considered an additional component and was spiked independently of the antibody concentrations. The components were therefore included in the following ranges: Tween™ 0.1%-1.0% and Adalimumab 0.1-1.0 g/L.

Calibration curves can be obtained as outlined in Example 6.1. The models developed above were used to generate predictions about spectra of mixtures of Tween™ and Adalimumab, in samples prepared according to the concentrations of Table 3:

TABLE 3

| Adalimumab (g/L) | Tween ™ (%) |
|---|---|
| 1.0 | 0.0 |
| 0.0 | 1.0 |
| 0.6 | 0.6 |
| 1.0 | 0.1 |
| 0.1 | 1.0 |
| 1.0 | 1.0 |
| 0.1 | 0.1 |
| 0.7 | 0.4 |
| 0.1 | 0.3 |
| 0.5 | 0.4 |
| 0.2 | 0.7 |
| 0.8 | 0.3 |

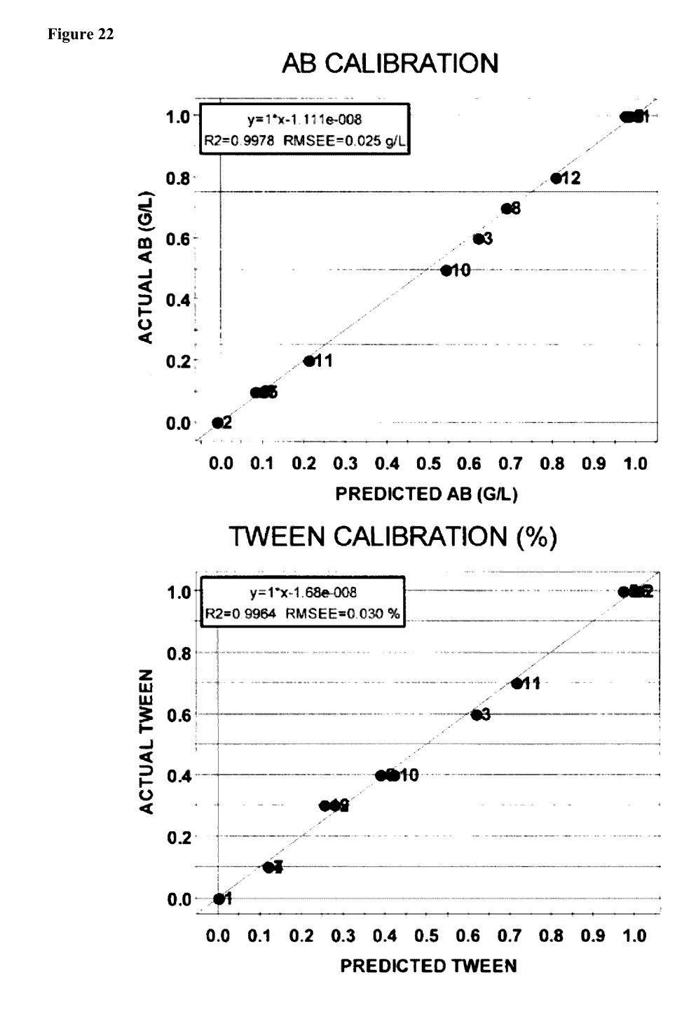

These predictions were compared against the actual spectra to confirm that the model falls within predetermined tolerances. The results are shown in FIG. 22A-B.

### 6.7 Testing of Antibody Aggregate Detection

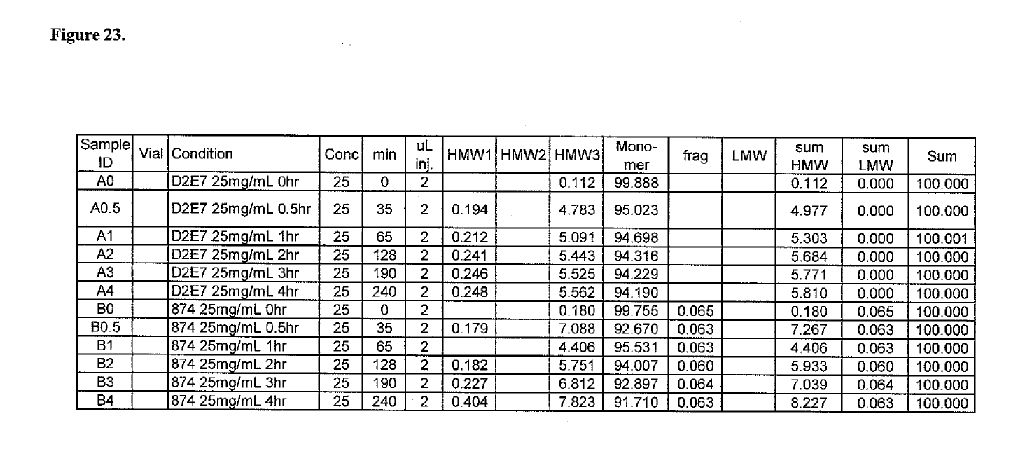

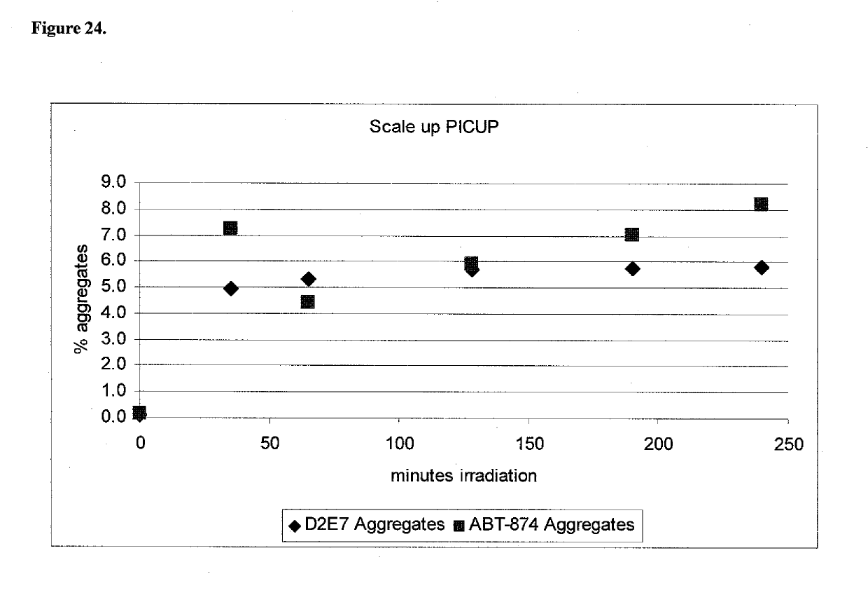

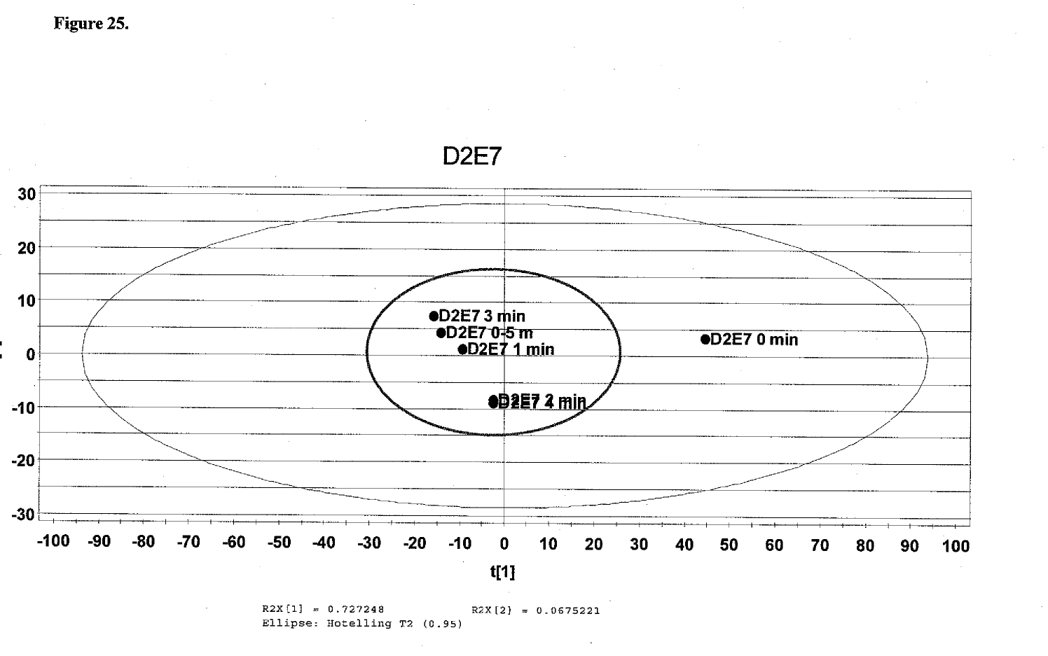

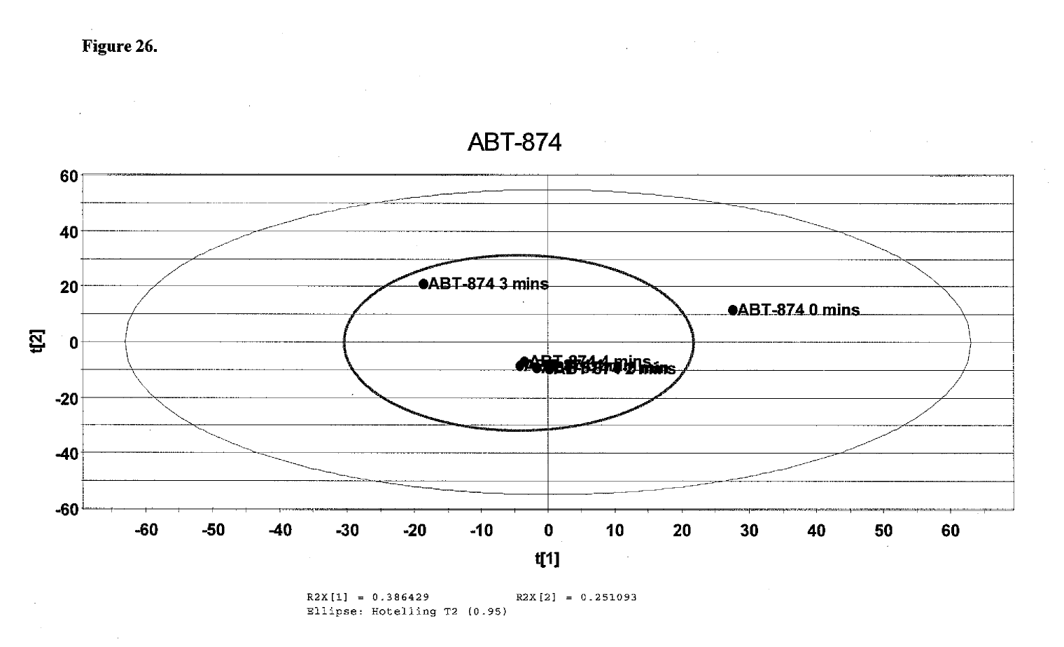

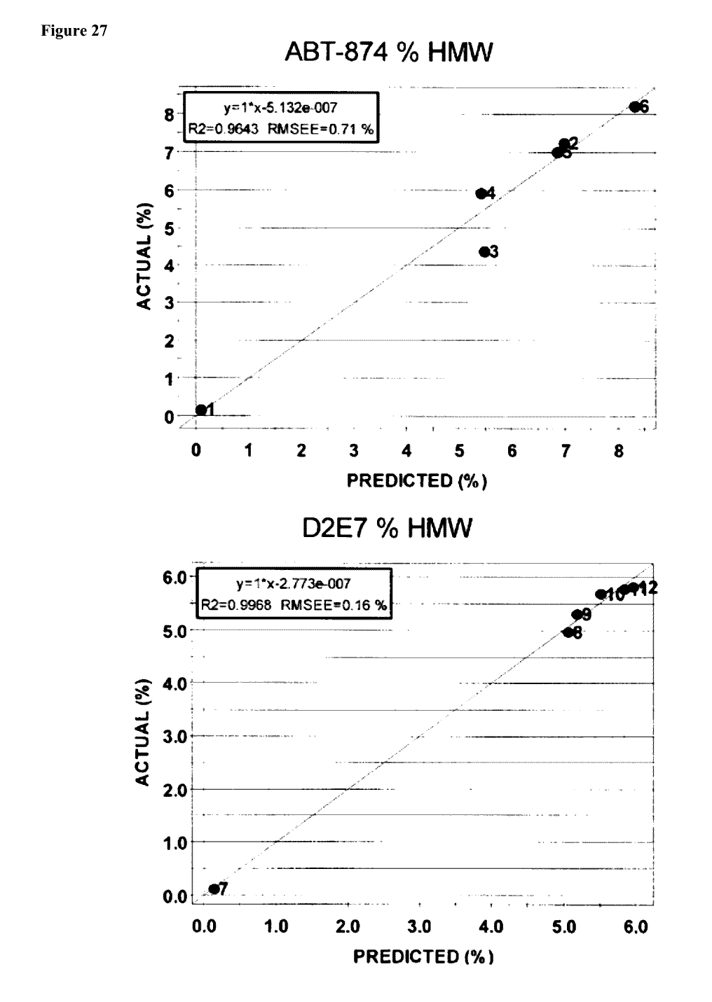

Two antibodies (D2E7 and ABT-874) were separately aggregated using photo induced cross linking of unmodified proteins (PICUP). The antibodies were exposed to the aggregating light source from 0-4 hours (FIGS. 23 and 24) and the aggregation quantified by size exclusion chromatography (SEC). Samples were measured by Raman spectroscopy and the spectra modeled using principal component analysis (PCA) (FIGS. 25 and 26) and partial least squares analysis (PLS) (FIGS. 27A and 27B). FIGS. 25 and 26 show that aggregated samples have distinct principal component scores and can be discriminated from aggregates using Raman spectroscopy. FIGS. 27A and 27B show some correlation between Raman spectroscopy results and the SEC measurements.

Various publications are cited herein, the contents of which are hereby incorporated by reference in their entireties.

What is claimed is:

1. A method of characterizing a multi-component mixture for use in a bioprocess operation comprising:
(a) providing a multi-component mixture standard with pre-determined amounts of known components;
(b) performing a Raman Spectroscopy analysis on the multi-component mixture standard;
(c) providing a multi-component test mixture from the bioprocess operation;
(d) performing a Raman Spectroscopy analysis on the multi-component test mixture; and
(e) comparing the analysis from step (d) with the analysis from step (b) to characterize the multi-component test mixture.

2. The method of claim 1, wherein the multi-component mixture standard and the multi-component test mixture both comprise one or more of a polysaccharide, an amino acid, and a pH buffer.

3. The method of claim 2, wherein the multi-component mixture standard and the multi-component test mixture both comprise at least two of a polysaccharide, an amino acid, a pH buffer.

4. The method of claim 3, wherein the polysaccharide is mannitol.

5. The method of claim 3 wherein the pH buffer is selected from a histidine and a citrate formulation.

6. The method of claim 3, wherein the multi-component mixture standard and the multi-component test mixture further comprises a surfactant.

7. The method of claim 6, wherein the surfactant is polysorbate 80.

8. The method of claim 1, comprising providing a series of multi-component mixture standards with pre-determined amounts of known components that are randomly selected, and performing a Raman Spectroscopy analysis on the series of multi-component mixture standards.

9. The method of claim 8, further comprising developing a model for characterizing the multi-component test mixture based on a Partial Least Squares Regression Analysis of the Raman Spectroscopy analysis on the series of multi-component mixture standards.

10. The method of claim 1, wherein the multi-component mixture is a formulation suitable for administration to an animal subject.

11. The method of claim 10, wherein the subject is a human.

12. The method of claim 11, wherein the formulation is to be combined with a biologically active agent, and wherein the biologically active agent and formulation, as combined, are approved by a regulatory authority.

13. The method of claim 1, wherein the multi-component mixture standard and the multi-component test mixture further comprises an agent selected from a monoclonal antibody, DNA, RNA, a protein, a virus, a virus subunit, a peptide and a vaccine.

14. The method of claim 13, wherein the multi-component mixture standard and the multi-component test mixture comprises a monoclonal antibody.

15. The method of claim 1, wherein the Raman Spectroscopy analysis on the multi-component test mixture is taken from an on-line sample from the bioprocess.

16. The method of claim 15, wherein the Raman Spectroscopy analysis is performed at regular intervals as part of a Quality Control procedure.

17. The method of claim 15, wherein the bioprocess is a filtration or purification operation.

18. The method of claim 1, wherein at least a portion of the multi-component mixture standard is added to the multicomponent text mixture.

19. The method of claim 2, wherein the multicomponent text mixture further comprises as least one of a tonicizer, a surfactant, a chelator, a salt, and an alcohol.

20. The method of claim 14, wherein the monoclonal antibody is adalimumab.
